(19) United States
(12) Patent Application Publication
(10) Pub. No.: US 2021/0193294 A1
(43) Pub. Date: Jun. 24, 2021

(54) PATIENT MANAGEMENT BASED ON SENSED ACTIVITIES

(71) Applicant: Hill-Rom Services, Inc., Batesville, IN (US)

(72) Inventors: Dee Anna Kumpar, Freeland, MI (US); Neal E. Wiggermann, Batesville, IN (US); Susan A. Kayser, Batesville, IN (US); Reyhaneh Sepehr, Fox Point, WI (US); Karrie Ann Schwencer, Batesville, IN (US)

(21) Appl. No.: 17/247,638

(22) Filed: Dec. 18, 2020

Related U.S. Application Data
(60) Provisional application No. 62/951,929, filed on Dec. 20, 2019.

(51) Int. Cl. G16H 20/30; G16H 40/63

(52) U.S. Cl. CPC G16H 20/30; G16H 40/63

(57) ABSTRACT

This disclosure is directed towards a patient management system for selectively generating, storing, and/or sharing patient activity data. A patient management system may receive, at an activity device, patient activity data indicative of activities of a patient. The patient management system may determine a raw score for the patient based on the activity data. The patient management system may combine component scores associated with activities performed by the patient to determine the raw score. Further, the patient management system may determine that the raw score is greater than a score threshold, and generate an activity score based on determining that the raw score is greater than the score threshold. The patient management system may output the activity score to an electronic device, such as a clinician device and/or a device associated with the patient, determine trends associated with the activity score, and/or compare the activity score to a mobility protocol.

### CROSS-REFERENCE TO RELATED APPLICATION

This application claims priority to U.S. Provisional Application No. 62/951,929, filed on Dec. 20, 2019 and entitled “PATIENT MANAGEMENT BASED ON SENSED ACTIVITIES,” the entirety of which is incorporated herein by reference.

### TECHNICAL FIELD

This application is directed to a patient management system, and in particular, to a system configured to selectively generate, store, and/or share patient activity data.

### BACKGROUND

Activities undergone by a person may affect health outcomes in a variety of ways. For example, mobilization of a patient may be associated with improved health outcomes, such as reducing a length of stay in a hospital and returning to normal activities faster than without mobilization. However, accurately monitoring mobilization and other activities of a patient can often present challenges, which may result in longer hospital stays, an increased number of doctor visits, and other negative outcomes for patients.

The various example embodiments of the present disclosure are directed toward overcoming one or more of the deficiencies associated with patient management systems.

### SUMMARY

Broadly, the systems and methods disclosed and contemplated herein are directed towards a patient management system for selectively generating, storing, and/or sharing patient activity data. In some examples, a computing device of a patient management system may receive, from a patient activity sensor, patient activity data indicative of activities of a patient. The patient management system may determine a raw score for the patient based at least in part on the activity data. For example, the patient management system may determine the raw score by identifying a first component score from the patient activity data, where the first component score is associated with a first activity of the activities of the patient. In some cases, the patient management system may determine a logarithm of the first component score associated with the first activity. Additionally, in some examples, the patient management system may identify a second component score from the activity data, where the second component score is associated with a second activity of the activities of the patient. The patient management system may combine the logarithm of the first component score with the second component score to determine the raw score for the patient. Further, the patient management system may determine that the raw score is greater than a score threshold, and generate an activity score based at least in part on determining that the raw score is greater than the score threshold. The patient management system may output the activity score to an electronic device, such as a clinician device and/or a device associated with the patient.

### BRIEF DESCRIPTION OF THE DRAWINGS

FIG. 1 shows a schematic block diagram of an example patient management system environment.

FIG. 2 shows a schematic block diagram of example activities that may be used by an example raw score component and/or example activity score component to generate a raw score and/or an activity score for a patient.

FIG. 3A is a diagram illustrating the use of an activity device to generate an assisted raw activity score and an unassisted raw activity score.

FIG. 3B is a diagram illustrating the use of an activity device to generate a raw score adjustment.

FIG. 4 is an example process for utilizing patient activity data to determine an activity score for a patient, according to the techniques described herein.

FIG. 5 is an example computing system and device which may be used to implement the described techniques.

### DETAILED DESCRIPTION

Various embodiments of the present disclosure will be described in detail with reference to the drawings, wherein like reference numerals represent like parts and assemblies throughout the several views. Additionally, any examples set forth in this specification are not intended to be limiting and merely set forth some of the many possible embodiments.

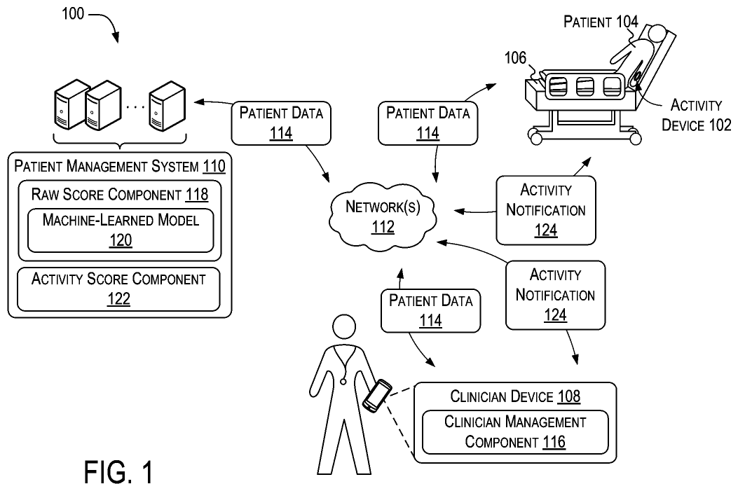

FIG. 1 shows a schematic block diagram of an example patient management system environment 100. The example patient management system environment 100 includes at least one activity device 102 (e.g., worn by a patient 104, where the patient 104 may also be referred to as a “wearer” of the wearable device 102), a healthcare establishment device 106 (e.g., a hospital bed), a clinician device 108, and a patient management system 110. The activity device 102, the healthcare establishment device 106, the clinician device 108, and/or the patient management system 110 may be in communication via one or more networks 112.

In some examples, the activity device 102 may be any suitable portable computing device that can store data and be transported by the patient 104, such as a watch, a necklace, a ring, a bracelet, eyeglasses, shoe(s), clothing, a patch, a belt, a band, and/or other type of accessory. Examples are also contemplated in which the activity device 102 comprises a phone, tablet, laptop computer, or other computing device that may not necessarily be “worn” on the body of the patient 104. In some cases, the activity device 102 may include one or more sensors, such as a heartrate sensor, respiration sensor, glucose sensor, blood pressure sensor, diagnostic sensor, motion sensor (e.g., accelerometer, gyroscope, etc.), and so forth.

In some examples, the healthcare establishment device 106 may be one of multiple healthcare establishment devices that generally exist in a healthcare establishment (e.g., doctor's office, hospital, clinic, dentist's office, pharmacy, ambulance, and the like) that may impact and/or monitor the health of the patient 104. For instance, the healthcare establishment device 106 may include a blood pressure device, an SpO₂ device, a temperature device, a respiratory device, a bodyweight scale, an otoscope, an ophthalmoscope, a stethoscope, a vision screening device, a hearing screening device, a microscope, an ECG device, an overhead lift device, a pressure-sensitive mat device, a bed and/or other furniture, a cane, a walker, and so on. In some instances, the healthcare establishment device 106 includes an accelerometer or other motion detection sensor to detect movement of the patient 104. Alternatively or additionally, the healthcare establishment device 106 may include a camera to generate images and/or video of an environment surrounding the healthcare establishment device 106.

In examples where the healthcare establishment device 106 is a hospital bed (or other type of furniture), the healthcare establishment device 106 may include load cells, air bladder pressure sensors, thermal sensors, pressure mapping sensors, ultrasonic sensors (e.g., to determine a distance of the patient 104 and/or a healthcare provider from the hospital bed), and the like. Further, in examples where the healthcare establishment device 106 is a hospital bed (or other type of furniture), the healthcare establishment device 106 may generate articulation data corresponding to an angle of the head and/or feet of the bed, which the healthcare establishment device 106 may use to determine a position or posture of the patient 104. While the healthcare establishment device 106 is described as existing within a healthcare establishment, examples are considered in which such devices may be found outside of a healthcare establishment, in some cases.

In examples, the clinician device 108 may include a computing device such as a mobile phone, a tablet computer, a laptop computer, a desktop computer, and so forth which provides a clinician (e.g., a doctor, nurse, technician, pharmacist, dentist, etc.) with information about the health of the patient 104. In some cases, the clinician device 108 may exist within a healthcare establishment (e.g., alongside the healthcare establishment device 106), although examples are also considered in which the clinician device 108 exists and/or is transported outside of a healthcare establishment, such as a doctor's mobile phone or home desktop computer that the doctor may use when the doctor is on-call. Alternatively or additionally, the clinician device 108 may include a device used in emergency medical situations (e.g., in an ambulance and/or accessible by emergency medical technicians (EMTs)), where the clinician devices in these situations can add, remove, change, and/or otherwise access data stored on the activity device 102.

The activity device 102, the healthcare establishment device 106, and/or the clinician device 108 may include a processor, microprocessor, and/or other computing device components, shown and described below. For instance, the activity device 102, the healthcare establishment device 106, and/or the clinician device 108 may be configured as mobile phones, tablet computers, laptop computers, etc., to deliver or communicate patient data 114 amongst one another and to other devices. In examples, the patient data 114 may include data associated with health of the patient 104, such as an electronic medical record (EMR) of the patient 104, along with (but not limited to) sensed inputs as described herein.

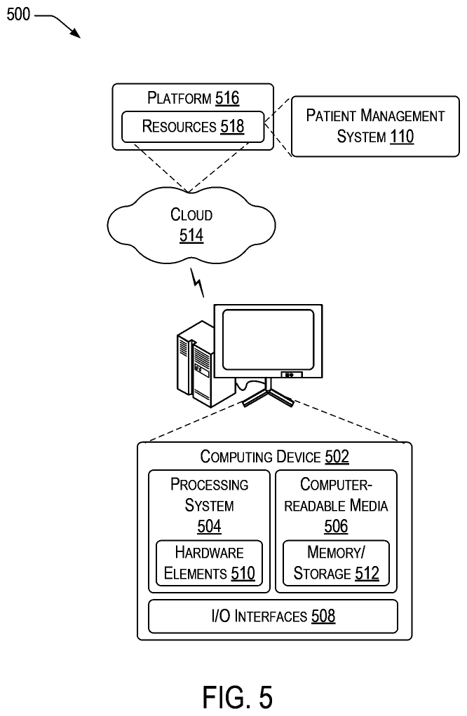

The patient management system 110 may be comprised of one or more server computing devices, which may communicate with the activity device 102, the healthcare establishment device 106, and/or the clinician device 108 to respond to queries, receive data, and so forth. Communication between the patient management system 110, the activity device 102, the healthcare establishment device 106, and/or the clinician device 108 occurs via the network 112, where the communication can include the patient data 114 related to the health of the patient 104. A server of the patient management system 110 can act on these requests from the activity device 102, the healthcare establishment device 106, and/or the clinician device 108, determine one or more responses to those queries, and respond back to activity device 102, the healthcare establishment device 106, and/or the clinician device 108. A server of the patient management system 110 may also include one or more processors, microprocessors, or other computing devices as discussed in more detail in relation to FIG. 5.

The patient management system 110 may include one or more database systems accessible by a server storing different types of information. For instance, a database can store correlations and algorithms used to manage the patient data 114 to be shared between the activity device 102, the healthcare establishment device 106, and/or the clinician device 108. A database can also include clinical data. A database may reside on a server of the patient management system 110 or on separate computing device(s) accessible by the patient management system 110.

The network 112 is typically any type of wireless network or other communication network known in the art. Examples of the network 112 include the Internet, an intranet, a wide area network (WAN), a local area network (LAN), and a virtual private network (VPN), cellular network connections and connections made using protocols such as 802.11a, b, g, n and/or ac. Alternatively or additionally, the network 112 may include a nanoscale network, a near-field communication network, a body-area network (BAN), a personal-area network (PAN), a near-me area network (NAN), a campus-area network (CAN), and/or an inter-area network (IAN).

In some examples, the patient management system 110, the activity device 102, the healthcare establishment device 106, and/or the clinician device 108 may generate, store, and/or selectively share the patient data 114 between one another to provide the patient 104 and/or clinicians treating the patient 104 with improved outcomes by providing a holistic picture of the activity of the patient 104. For instance, the activity device 102 and/or the healthcare establishment device 106 may sense an activity associated with the patient 104, such as based on movement, heart rate, respiratory rate, blood pressure, and so forth, and store patient data 114 in the form of values associated with the activity for at least a period of time. The period of time may be a predetermined time (e.g., one minute, one hour, one day, etc.), or a variable time (e.g., between visits by a clinician to a hospital room of the patient, until stopped by the patient 104 and/or a clinician, etc.).

In some cases, the activity device 102 and the healthcare establishment device 106 may communicate with one another to, among other things, verify the occurrence of an activity of the patient 104. For example, the healthcare establishment device 106 (in this example, a hospital bed) may detect a change in weight on the surface of the hospital bed, which indicates a person sitting on a side of the hospital bed. Before generating activity data corresponding to the patient 104 sitting on the side of the hospital bed, the healthcare establishment device 106 may verify a location of the patient in relation to the hospital bed 106 with the activity device 102 and/or via an image or video of an environment surrounding the healthcare establishment device 106 provided by a camera in the environment. If the location of the patient 104 is verified by the activity device 102 (or the image or video) to be within a threshold distance (e.g., 1 foot, 1 meter, 2 meters, etc.) of the hospital bed, the hospital bed (and/or the activity device 102) may generate activity data corresponding to the activity of the patient 104 sitting on the side of the hospital bed. In some examples, a computer vision system may use images and/or video to determine if an activity, such as ambulating or turning, is within the presence of a clinician (e.g., by determining that the clinician is less than a threshold distance from the patient 104 in the image, such as less than 1 meter) or more than one clinician. In such examples, an indication that an activity took place in the presence of a clinician may be provided to a raw score component 118 of the patient management system 110 for use in determining whether the activity was assisted or unassisted in determining a raw score for the activity. Additional information related to determining whether a clinician is assisting with an activity using a computer vision system may be found in relation to “A computer vision system for deep learning-based detection of patient mobilization activities in the ICU,” Yeung et al., Nature Partner Journals, npj Digit. Med. 2, 11 (2019), which is incorporated by reference herein in its entirety.

However, in some cases, other people (e.g., other than the patient 104) may sit on the hospital bed, which in conventional systems may cause inaccurate activity data to be generated for the patient 104. Therefore, if the location of the patient 104 is verified by the activity device 102 to be outside of the threshold distance of the hospital bed, the hospital bed (and/or the activity device 102) may prevent the activity data corresponding to the patient 104 sitting on the side of the hospital bed from being generated. In some cases, the healthcare establishment device 106 may use the image and/or video to verify the occurrence of the activity using computer vision, such as by identifying the activity device 102 within or outside of the threshold distance in an image or video, using facial recognition of the patient 104 and determining whether the face of the patient is within or outside of the threshold distance in an image or video, and so forth. Alternatively or additionally, the activity device 102, the healthcare establishment device 106, and/or the clinician device 108 may use sensed biological parameters of the patient 104 to verify the occurrence of an activity. For example, the activity device 102 may detect that the patient 104 is moving, but the healthcare establishment device 106 may not detect an increase in heart rate of the patient. In this illustrative example, the absence of an increase in heart rate may cause the activity device 102, the healthcare establishment device 106, and/or the clinician device 108 to prevent the motion from being labeled as unassisted ambulating by the patient 104, as unassisted ambulating often results in increased heart rate.

In another illustrative example, the activity device 102 may receive an indication from a first healthcare establishment device 106 (e.g., a hospital bed) that the patient 104 has exited the hospital bed. In this case, the activity device 102 may also receive an indication from a second healthcare establishment device 106 (e.g., an overhead lift sensor) indicating whether the patient 104 used the overhead lift to exit the hospital bed. The activity device 102 may verify that the patient 104 exited the hospital bed, and how the patient 104 exited the hospital bed, using information received from both the first healthcare establishment device and the second healthcare establishment device. Additional examples of verifications that may take place between the activity device 102 and the healthcare establishment device 106 may include: determining a posture of the patient 104 (e.g., prone, supine, left, right, recline, etc.); differentiating between the patient 104 sitting up in bed, sitting on the side of the bed, and/or sitting standing outside of the bed; determining whether the patient 104 is ambulating, how many ambulating steps the patient 104 took, how long the patient 104 ambulated (e.g., number of seconds, minutes, etc.), and so forth.

Alternatively or additionally, the clinician device 108 may communicate with one or both of the activity device 102 and the healthcare establishment device 106 to verify the occurrence of an activity of the patient 104. In particular, the clinician device 108 may determine a proximity to the activity device 102 and/or the healthcare establishment device 106, and in turn, a clinician management component 116 of the clinician device 108 may determine whether an activity of the patient 104 was assisted or unassisted by a clinician. In one illustrative example, the activity device 102 and/or the healthcare establishment device 106 may receive real-time location service (RTLS) data from the clinician device 108, which may indicate a proximity of the clinician device 108 to the activity device 102 and/or the healthcare establishment device 106. Alternatively or additionally, a camera of the healthcare establishment device 106 and/or the clinician device 108 may provide an image or video to one or both of the devices, which may indicate a proximity of the clinician device 108 to the activity device 102 and/or the healthcare establishment device 106. Using the RTLS and/or image or video data, the activity device 102 and/or the healthcare establishment device 106 may determine whether a clinician associated with (e.g., carrying) the clinician device 108 is within a threshold distance of the patient 104. If the clinician device 108 is within the threshold distance of the activity device 102 and/or the healthcare establishment device 106, the activity device 102 and/or the healthcare establishment device 106 may determine that an activity, such as a turn in a hospital bed, is assisted. On the other hand, if the clinician device 108 is outside of the threshold distance of the activity device 102 and/or the healthcare establishment device 106, the activity device 102 and/or the healthcare establishment device 106 may determine that the activity is unassisted.

Additionally, examples are considered where the patient 104 and/or a clinician manually inputs an activity into the activity device 102, the healthcare establishment device 106, and/or the clinician device. For example, the clinician management component 116 may receive a mobility protocol for the patient 104 from the patient management system 110, where the mobility protocol includes mobility exercises that the patient 104 is to perform. Upon completion of a mobility exercise (or a portion thereof) included in the mobility protocol, the patient 104 may input to the activity device 102 that the mobility exercise has been completed or partially completed. Alternatively or additionally, upon completion of a mobility exercise (or a portion thereof) included in the mobility protocol, a clinician may input to the clinician management component 116 of the clinician device 108 that the mobility exercise has been completed or partially completed. The activity device 102, the healthcare establishment device 106, and/or the clinician device may include the mobility exercises, as completed or partially completed, as activities in the patient data 114.

The activity device 102, the healthcare establishment device 106, and/or the clinician device 108 may generate a variety of patient activity data to be included in the patient data 114. For instance, the activity device 102, the healthcare establishment device 106, and/or the clinician device 108 may indicate activities in the patient data 114 such as assisted steps, unassisted steps, assisted turns in a hospital bed, unassisted turns in a hospital bed, assisted exits from the hospital bed, unassisted exits from the hospital bed, assisted ambulating, unassisted ambulating, sitting in the hospital bed, sitting out of the hospital bed, and/or limb or extremity movement, among others. In some examples, the activity device 102, the healthcare establishment device 106, and/or the clinician device 108 may use an EMR included in the patient data 114 to distinguish between sensed inputs. For instance, the EMR included in the patient data 114 may indicate that the patient 104 is using a wheelchair, and thus the activity device 102, the healthcare establishment device 106, and/or the clinician device 108 may categorize movement by the patient 104 throughout an environment as assisted ambulating rather than unassisted ambulating based on this information in the EMR.

In some examples, the activity device 102, the healthcare establishment device 106, and/or the clinician device 108 may output the patient data 114 including data related to activities of the patient 104 to the patient management system 110. The patient management system 110 may include a raw score component 118 that determines a raw score for the patient 104 based at least in part on the patient data 114 received from the activity device 102, the healthcare establishment device 106, and/or the clinician device 108. As described above and in more detail below, the raw score component 118 may determine the raw score by identifying a first component score from the patient data 114, where the first component score is associated with a first activity of multiple activities detected in relation to the patient 104. In some cases, the raw score component 118 may determine a logarithm of the first component score associated with the first activity.

For instance, using a logarithm of a component score may give more weight to the component score associated with a particular activity when the activity is performed fewer times. In an illustrative example, taking a logarithm of a component score associated with unassisted steps by the patient 104 will indicate greater progress achieved by the patient 104 when the patient takes 4 unassisted steps in one day, than when the patient takes 84 unassisted steps in one day. In this way, clinicians and patients may have a better picture of progress achieved by the patients in different stages of recovery.

Additionally, in some cases, the raw score component 118 may identify a second component score associated with a second activity of the patient 104, a third component score associated with a third activity of the patient 104, and so forth, for as many activities as are recorded by the activity device 102, the healthcare establishment device 106, and/or the clinician device 108. The raw score component 118 may combine the logarithm of the first component score with the second component score, the third component score, and so forth to determine a raw score for the patient 104 over a time period (e.g., one hour, eight hours, twelve hours, one day, one week, etc.), such as by adding the component scores, taking an average of the component scores, and so on.

In some examples, the raw score component 118 may weight one or more of the component scores when combining the component scores together. For instance, the raw score component 118 may weight the component scores based, in part, on an intensity level of the activities associated with the component scores. In one illustrative example, a component score associated with a number of minutes spent sitting in a chair will have a lower weight than a component score associated with a number of minutes ambulating. In some cases, the raw score component 118 may receive weight values to assign to activities associated with component scores from the clinician management component 116 of the clinician device 108. For example, the clinician management component 116 may enable individual clinicians to customize weight values for different activities based on standards set by a particular healthcare establishment, according to a mobility protocol for the patient 104, and so forth.

Additionally, in some cases, the raw score component 118 may determine a maximum threshold number for a particular activity that, when the activity corresponding to the threshold is exceeded by the patient 104, does not count towards the corresponding component score. For instance, if the patient 104 ambulates for more than 60 minutes and the threshold score for ambulating is 30 minutes, the raw score component 118 will disregard the remaining 30 minutes of the patient ambulating beyond the threshold. The threshold(s) may be set by a clinician and input to the clinician management component 116 for different activities, similar to the discussion of weights above.

Alternatively or additionally, the raw score component 118 may include a machine-learned model 120 trained to determine weight values for different activities, such as based on intensity levels of the activities. For example, the machine-learned model 120 may include an artificial neural network, a decision tree, a regression algorithm, or other machine-learning algorithm to determine weight values for different activities. In some examples, the machine-learned model 120 may also determine which of the component scores of corresponding activities to apply a logarithm to, and what base of the logarithm to apply the values associated with particular activities. Additionally, in some cases, the machine-learned model 120 may determine which of the component scores of corresponding activities to apply a maximum threshold to, and what the maximum threshold value should be for the particular activity. The raw score component 118 may use weight values, logarithms, and/or thresholds received from the machine-learned model 120 to determine the raw score for the patient 104.

In some instances, the raw score component 118 provides a raw score (e.g., as part of the patient data 114) to the clinician device 108. The clinician device 108 may display the raw score to a healthcare provider in a user interface so that the healthcare provider can view how the raw score was calculated, make corrections to inputs to the raw score (e.g., by modifying an activity type of a sensed activity by the patient 104), and the like. For example, the healthcare provider may select an indication of the raw score in the user interface, and in response the clinician device 108 displays what activities were tracked by the activity device 102, the healthcare establishment device 106, and/or the clinician device 108, and used to generate the raw score.

In some examples, the raw score component 118 may output a raw score determined from various component scores for the patient 104 to an activity score component 122. The activity score component 122 may compare the raw score to one or more threshold scores, where the one or more threshold scores correspond different activity scores that may be assigned to the patient 104. For example, consider the following table of raw score ranges that may correspond to respective activity scores:

| Raw Score Range | Activity Score |
|---|---|
| 0 | 0 |
| 1-10 | 1 |
| 11-50 | 2 |
| 51-60 | 3 |
| 61-70 | 4 |
| 71-100 | 5 |
| 101-150 | 6 |
| 151-200 | 7 |
| 201-250 | 8 |
| 251-300 | 9 |
| >301 | 10 |

In some examples, the activity score component 122 may output an activity score as part of an activity notification 124 for the patient 104 (e.g., to the clinician device 108, the activity device 102, and/or other devices) at regular intervals, such as each hour, every 8 hours, each day, each week, and so forth.

In some cases, the raw score component 118 and/or the activity score component 122 may pause collection of the activity data included in the patient data 114, or pause generation of the raw score or the activity score. For instance, consider a scenario in which the patient 104 goes into surgery to have a procedure completed. The patient 104 and/or a clinician may pause collection of activity data during the surgery to prevent the activity score for the patient 104 from being skewed during the time of the surgery. Alternatively or additionally, the activity device 102 may detect a change in location of the patient 104 (e.g., by leaving the patient's hospital room, entering a surgery center, etc.), and either automatically pause collection of the activity data, or output an activity notification 124 to the clinician device 108 asking the clinician whether the collection of activity data should be paused. The raw score component 118 may determine the raw score for the patient 104 exclusive of the time during which collection of the activity data has been paused, thus giving an accurate representation of patient activity during times when the patient 104 is able to perform the various activities.

Additionally, in some examples, the raw score component 118 may selectively include activities in the activity data used to generate the raw score based on verifications received from the clinician device 108. For example, the activity device 102 may detect an activity being performed by the patient 104, such as the patient ambulating, and output the activity data to the patient management system 110. Prior to including the activity data in the raw score, the raw score component 118 may output an activity notification 124 to the clinician device 108 that the patient 104 is performing the ambulating activity, and asking a clinician to verify the ambulating activity. The raw score component 118 may receive a verification from the clinician device 108, and responsive to receiving the verification, may include the ambulating activity in the raw score. On the other hand, the raw score component 118 may receive an indication from the clinician device that the patient 104 is not performing the ambulating activity, and/or not receive a response from the clinician device 108. In either one of these scenarios, the raw score component 118 may exclude the ambulating activity from the raw score.

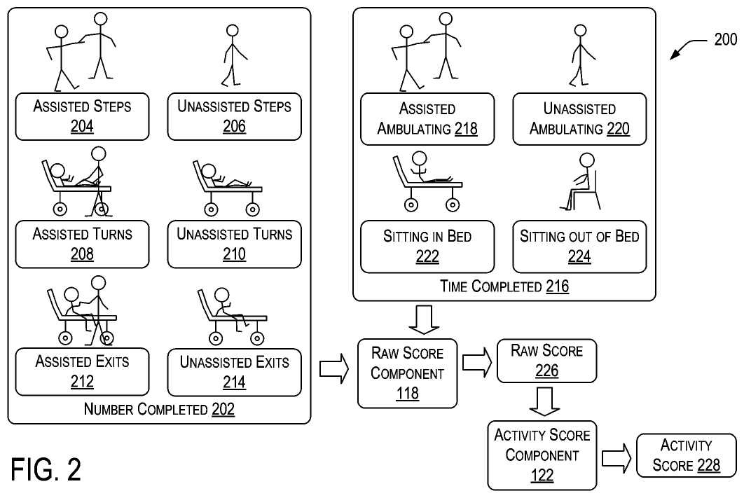

Example configurations of the activity device 102, the healthcare establishment device 106, and/or the clinician device 108, and methods for their use, are shown and described with reference to at least FIGS. 2-5 below.

FIG. 2 shows a schematic block diagram 200 of example activities that may be used by an example raw score component and/or example activity score component to generate a raw score and/or an activity score for a patient. In some examples, the activity device 102, the healthcare establishment device 106, and/or the clinician device 108 may detect and/or verify the occurrence of any of the described activities, as discussed above and below. For instance, the activity device 102, the healthcare establishment device 106, and/or the clinician device 108 may detect number completed activities 202, which may correspond to a count (e.g., an integer of 1, were the integer is equal to or greater than 0) associated with a number of times the respective activity was completed by the patient 104. Alternatively or additionally, the activity device 102, the healthcare establishment device 106, and/or the clinician device 108 may detect presence or absence of a particular activity, where presence of an activity may result in a score of 1 and absence of an activity may result in a score of 0 (e.g., a Boolean score for an activity).

The number completed activities 202 may include assisted steps 204, which may correspond to steps taken by the patient 104 with assistance from a clinician, assistance from a walking assistive device (e.g., crutches, walker, parallel bar(s), etc.) and the like. Likewise, the number completed activities 202 may include unassisted steps 206, which may correspond to steps taken by the patient 104 without assistance from a clinician, assistance from a walking assistive device (e.g., crutches, walker, parallel bar(s), etc.) and the like. In examples, the activity device 102 may detect that the patient 104 is taking steps, and may send an activity notification 124 to the clinician device 108 and/or the activity device 102 to request a verification that the steps are assisted or unassisted.

In some examples, the number completed activities 202 may include assisted turns 208, which may correspond to turns taken by the patient 104 in a hospital bed (e.g., the healthcare establishment device 106 as discussed above) with assistance from a clinician. The number completed activities 202 may also include unassisted turns 210, which may correspond to turns taken by the patient 104 in a hospital bed (e.g., the healthcare establishment device 106 as discussed above) without assistance from a clinician. Similar to above, the activity device 102 and/or the healthcare establishment device 106 may detect that the patient 104 has turned in the hospital bed, and may send an activity notification 124 to the clinician device 108 and/or the activity device 102 to request a verification that the turn was assisted or unassisted.

Additionally, in some cases, the number completed activities 202 may include assisted exits 212, which may correspond to exits taken by the patient 104 from a hospital bed (e.g., the healthcare establishment device 106 as discussed above) with assistance from a clinician, and/or assistance from another healthcare establishment device such as an overhead lift. The number completed activities 202 may also include unassisted exits 214, which may correspond to exits taken by the patient 104 from a hospital bed (e.g., the healthcare establishment device 106 as discussed above) without assistance from a clinician, and/or assistance from another healthcare establishment device such as an overhead lift. Similar to above, the activity device 102 and/or the healthcare establishment device 106 may detect that the patient 104 has exited from the hospital bed, and may send an activity notification 124 to the clinician device 108 and/or the activity device 102 to request a verification that the exit was assisted or unassisted.

In some examples, the activity device 102, the healthcare establishment device 106, and/or the clinician device 108 may detect time completed activities 216, which may correspond to an amount of time (e.g., minutes, seconds, hours, etc.) that the patient 104 spent performing an activity. For instance, the time completed activities 216 may include assisted ambulating 218, which may correspond to an amount of time that the patient 104 spent ambulating with assistance from a clinician, assistance from a walking assistive device (e.g., crutches, walker, parallel bar(s), etc.) and the like. Likewise, the time completed activities 216 may include unassisted ambulating 220, which may correspond to an amount of time that the patient 104 spent ambulating without assistance from a clinician, assistance from a walking assistive device (e.g., crutches, walker, parallel bar(s), etc.) and the like. In examples, the activity device 102 may detect that the patient 104 is taking steps (or otherwise ambulating), and may send an activity notification 124 to the clinician device 108 and/or the activity device 102 to request a verification for an amount of time ambulating that was assisted or unassisted.

The time completed activities 216 may further include sitting in bed 222, which may correspond to an amount of time that the patient 104 spent sitting in a hospital bed (e.g., the healthcare establishment device 106), such as propped by a backrest of the hospital bed, sitting upright without being propped by the backrest, sitting on a side of the hospital bed, and the like. Likewise, the time completed activities 216 may include sitting out of bed 224, which may correspond to an amount of time that the patient 104 spent sitting outside of a hospital bed (e.g., the healthcare establishment device 106), such as in a chair, in a wheelchair, on a physical therapy device (e.g., a therapy ball), and so forth. In examples, the activity device 102 and/or the healthcare establishment device 106 may detect that the patient 104 is taking sitting, and may send an activity notification 124 to the clinician device 108 and/or the activity device 102 to request a verification for an amount of time that the patient 104 was sitting in bed and/or sitting out of bed. The number completed activities 202 and the time completed activities 216 are intended only as examples of activities that the raw score component 118 may use to determine a raw score for the patient 104, and are not meant to be limiting. For instance, although not explicitly pictured, the number completed activities 202 and/or the time completed activities 216 may include limb or extremity movement, such as where the patient moves an arm or a leg, but may not traverse a portion of the environment (e.g., as part of a physical therapy program).

As discussed above, the raw score component 118 receives patient data 114 that may include activity data related to the number completed activities 202 and/or the time completed activities 216. The raw score component 118 may use the activity data to generate a raw score 226 for the patient 104. For instance, the raw score component 118 may weight one or more of the values corresponding to the number completed activities 202 and/or the time completed activities 216 included in the raw score 226. In one illustrative example, the raw score component 118 may weight a value corresponding to the sitting out of bed 224 performed by the patient 104 by multiplying the value by 1.5, and may weight a value corresponding to the sitting in bed 222 performed by the patient 104 by multiplying the value by 1. The raw score component 118 may apply weights to the values of the activities based on an intensity of the respective activities, such that more intense activities impact the raw score more so than less intense activities.

Alternatively or additionally, the raw score component 118 may take a logarithm of one or more values corresponding to the number completed activities 202 and/or the time completed activities 216 included in the raw score 226. In an illustrative example, the raw score component 118 may take a logarithm (e.g., base 10) of a number of the assisted turns 208 and/or a number of the unassisted turns 210. In this way, an increased number of the assisted turns 208 and/or an increased number of the unassisted turns 210 impacts the raw score 226 less dramatically as the individual number of turns by the patient 104 increases. This may allow other activities that may be more intense than turns in a hospital bed to have a greater impact on the raw score, thus more accurately reflecting the activity of the patient 104.

Further, the raw score component 118 exclude one or more values corresponding to the number completed activities 202 and/or the time completed activities 216 from being included in the raw score 226 if the values are above a threshold amount. As discussed above, if the patient 104 ambulates for more than 60 minutes and the threshold score for ambulating is 30 minutes, the raw score component 118 will disregard the remaining 30 minutes of the patient ambulating beyond the threshold. This may enable the raw score component 118 to account for other activities in the raw score 226 that would otherwise be overwhelmed by the amount of the particular activity performed by the patient.

As discussed above, the activity score component 122 may receive the raw score 226, and may output an activity score 228 based on the raw score 226, such as to the clinician device 108 and/or the activity device 102. The activity score component 122 may determine the activity score 228 by comparing the raw score 226 to one or more threshold scores, where the one or more threshold scores correspond different activity scores that may be assigned to the patient 104, as described above. In some cases, the activity score component 122 may require particular activities to have a minimum value in order to output a minimum activity score 228. For example, if the patient 104 does not spend any time sitting in bed, sitting out of bed, or ambulating, then the activity score component 122 may output an activity score of 0, regardless of a number of turns (assisted or unassisted) or bed exits performed by the patient 104.

In addition to outputting the activity score 228 itself to the clinician device 108 and/or the activity device 102, the activity score component 122 may output trends associated with the patient's activity scores over time. For instance, the trends may include how the patient's activity scores have changed over the course of a day, over the course of a week, over the course of a month, and so forth. In some cases, the activity score component 122 may also output values and/or trends associated with activities used to determine the activity score 228, such as individual ones of the number completed activities 202 and/or the time completed activities 216.

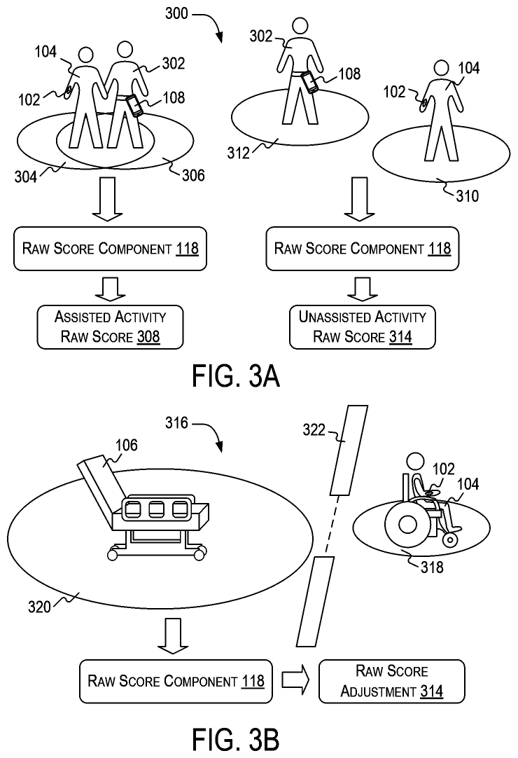

FIG. 3A is a diagram 300 illustrating the use of an activity device to generate an assisted raw activity score and an unassisted raw activity score. The diagram 300 includes the patient 104 wearing the activity device 102, and a clinician 302 wearing the clinician device 108. In some examples, the activity device 102 may output a location of the patient 104 to the clinician device 108. Alternatively or additionally, the clinician device 108 may output a location of the clinician 302 to the activity device 102. The activity device 102 and/or the clinician device 108 may determine a threshold distance 304 surrounding the patient 104, and a threshold distance 306 surrounding the clinician 302. The threshold distance 304 and/or the threshold distance 306 may be, for instance, a 1-foot radius, a 2-foot radius, a 3-foot radius, and so forth.

Additionally, the activity device 102 and/or the clinician device 108 may determine that the threshold distance 304 and the threshold distance 306 overlap with one another. Based on this determination, the activity device 102 and/or the clinician device 108 may output an indication to the raw score component 118 that the activity being performed by the patient 104 (in this example, ambulating or taking steps) is being assisted by the clinician 302. The raw score component 118 may generate an assisted activity raw score 308 based on the determination that the activity being performed by the patient 104 is being assisted by the clinician 302.

In some cases, activity device 102 and/or the clinician device 108 may determine a threshold distance 310 surrounding the patient 104, and a threshold distance 312 surrounding the clinician 302. The threshold distance 310 and/or the threshold distance 312 may be, for instance, a 1 foot radius, a 2 foot radius, a 3 foot radius, and so forth. In the illustrated example, the threshold distance 310 surrounding the patient 104 and the threshold distance 312 surrounding the clinician 302 do not overlap with one another. The activity device 102 and/or the clinician device 108 may determine that the threshold distance 310 and the threshold distance 312 do not overlap, and may output an indication to the raw score component 118 that the activity being performed by the patient 104 (in this example, ambulating or taking steps) is not being assisted by the clinician 302. The raw score component 118 may generate an unassisted activity raw score 314 based on the determination that the activity being performed by the patient 104 is not being assisted by the clinician 302. In examples, the raw score component 118 may use the assisted activity score 308 and/or the unassisted activity score 314 to generate a raw score 226 for the patient 104, as described above.

FIG. 3B is a diagram 316 illustrating the use of activity device to generate a raw score adjustment. The diagram 316 includes the patient 104 wearing the activity device 102, and the healthcare establishment device 106, in this case a hospital bed. In some examples, the activity device 102 may output a location of the patient 104 to the healthcare establishment device 106. Alternatively or additionally, the healthcare establishment device 106 may output a location of the healthcare establishment device 106 to the activity device 102. The activity device 102 and/or the healthcare establishment device 106 may determine a threshold distance 318 surrounding the patient 104, and a threshold distance 320 surrounding the healthcare establishment device 106. The threshold distance 318 and/or the threshold distance 320 may be, for instance, a 1-foot radius, a 5-foot radius, a 10-foot radius, and so forth. Alternatively or additionally, the activity device 102 may determine a location of the patient 104 relative to a landmark 322, such as a boundary associated with a hospital room assigned to the patient 104 that contains the healthcare establishment device 106.

In some examples, the activity device 102 and/or the healthcare establishment device 106 may determine that the threshold distance 318 and the threshold distance 320 do not overlap with one another. For instance, the patient 104 may be being taken to surgery, and during this time, may not be capable of performing activities considered in the activity data. Therefore, in some cases, a clinician may desire for activity data collection to be paused (or be removed from analysis for a time), to prevent an activity score for the patient 104 from being skewed because of the activity.

For example, the activity device 102 and/or the healthcare establishment device 106 may output an activity notification 124 to the raw score component 118 that a threshold distance between the activity device and the healthcare establishment device 106 has been exceeded (e.g., the threshold distance 318 and the threshold distance 320 do not overlap). In some cases, the activity device 102 and/or the healthcare establishment device 106 may, based on this determination, automatically cease collection of activity data. Alternatively or additionally, the raw score determination component 118 may exclude data collected by the activity device 102 and/or the healthcare establishment device 106 for as long as the threshold distance between the devices is exceeded. In some examples, the raw score component 118 may output a notification to the clinician device 118 and/or the activity device 102 to verify that data collection should be paused, before excluding the activity data during this time. For instance, the patient 104 may be being taken on a walk by a family member throughout the hospital, and thus the clinician may not want data collection to be ceased during this time. Therefore, the notification may provide the clinician with an option to exclude the data before the data is excluded.

The raw score component 118 may generate a raw score adjustment 324 based on a time during which the threshold distance 318 and the threshold distance 320 do not overlap. For example, the raw score adjustment 324 may exclude data collected by the activity device 102 and/or the healthcare establishment device 106 from the raw score 226 during the time when the threshold distance 318 and the threshold distance 320 do not overlap. In some cases, the raw score component 118 may continue to include activity data in determining the raw score 226 during the time when the threshold distance 318 and the threshold distance 320 do not overlap until a verification is received from the clinician device 108 to exclude data during this time.

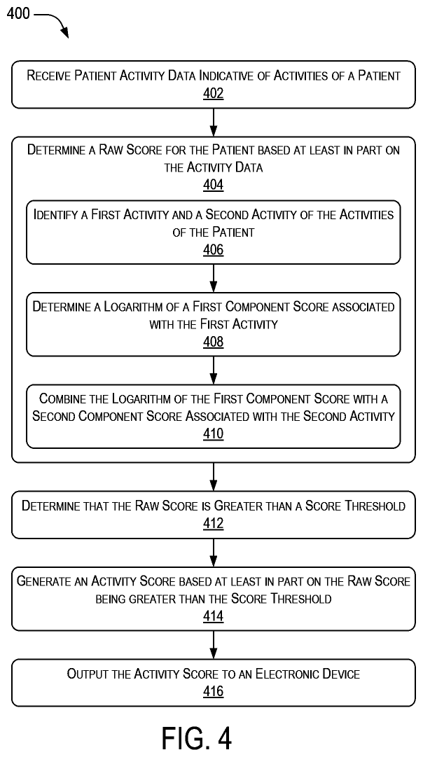

FIG. 4 is an example process 400 for utilizing patient activity data to determine an activity score for a patient, according to the techniques described herein. In some examples, the process 400 may be performed by one or more processors of computing devices, such as the activity device 102 of FIG. 1.

At operation 402, the process can include receiving, by one or more processors of a patient management system, patient activity data indicative of activities of a patient. For instance, the patient management system 110 may receive patient activity data from the activity device 102, the healthcare establishment device 106, and/or the clinician device 108 corresponding to activities performed by the patient 104. The activities of the patient 104 may include, but are not limited to, one or more of the number completed activities 202 and the time completed activities 216 described above.

At operation 404, the process can include determining, by the one or more processors, a raw score for the patient based at least in part on the activity data. In some examples, determining the raw score for the patient may include, at operation 406, identifying, by the one or more processors, a first activity and a second activity of the activities of the patient. In some cases, identifying the activities may include the raw score component 118 determining whether the first activity and/or the second activity are assisted or unassisted. Determining the raw score for the patient may also include, at operation 408, determining, by the one or more processors, a logarithm of a first component score associated with the first activity. For instance, the logarithm may dampen a number or an amount of time of a particular activity as the number or the amount of time gets larger. Thus, smaller numbers of the activity may have a greater impact on an overall activity score for the patient when a logarithm is applied than larger numbers of the activity. Additionally, determining the raw score for the patient may include at operation 410, combining, by the one or processors, the logarithm of the first component score with a second component score associated with the second activity. In some examples, the raw score component 118 may weight the logarithm of the first component score and/or the second component score based on an intensity of the respective activities, to give more intense activities more influence on the activity score for the patient 104. The raw score component 118 may combine the logarithm of the first component score with the second component score by adding the two together, in one example.

At operation 412, the process can include determining, by the one or more processors, that the raw score is greater than a score threshold. For instance, the activity score component 122 may compare the raw score to one or more threshold scores, where the one or more threshold scores correspond different activity scores that may be assigned to the patient 104. If the raw score falls within a range of scores corresponding to a particular activity score, the patient 104 may be assigned the corresponding activity score for an associated time period (e.g., one hour, eight hours, one day, etc.). For example, at operation 414, the process can include generating, by the one or more processors, an activity score based at least in part on the raw score being greater than the score threshold.

At operation 416, the process can include outputting, by the one or more processors, the activity score to an electronic device, such as the activity device 102 and/or the clinician device 108. In some examples, the activity score component 122 may also provide, based at least in part on the activity score, a recommended change to a mobility assessment in an EMR of the patient. For instance, if the activity score has continued to increase, the activity score component 122 may recommend more complex activities for the patient 104 to perform, such as going from assisted ambulating to unassisted ambulating. In another example, the activity score component 122 may recommend, with the activity score, changes to an amount of assistance that the patient 104 is receiving. For instance, the activity score component 122 may recommend transitioning from two clinicians assisting with an activity to one clinician assisting with the activity being performed by the patient 104.

FIG. 5 is an example computing system and device which may be used to implement the described techniques.

### Example System and Device

FIG. 5 illustrates an example system generally at 500 that includes an example computing device 502 that is representative of one or more computing systems and/or devices that may implement the various techniques described herein. This is illustrated through inclusion of the patient management system 110. The computing device 502 may be, for example, a server of a service provider, a device associated with a client (e.g., a client device), an on-chip system, and/or any other suitable computing device or computing system.

The example computing device 502 as illustrated includes a processing system 504, one or more computer-readable media 506, and one or more I/O interface 508 that are communicatively coupled, one to another. Although not shown, the computing device 502 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 504 is representative of functionality to perform one or more operations using hardware. Accordingly, the processing system 504 is illustrated as including hardware element 510 that may be configured as processors, functional blocks, and so forth. This may include implementation in hardware as an application specific integrated circuit or other logic device formed using one or more semiconductors. The hardware elements 510 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable storage media 506 is illustrated as including memory/storage 512. The memory/storage 512 represents memory/storage capacity associated with one or more computer-readable media. The memory/storage component 512 may include volatile media (such as random access memory (RAM)) and/or nonvolatile media (such as read only memory (ROM), Flash memory, optical disks, magnetic disks, and so forth). The memory/storage component 512 may include fixed media (e.g., RAM, ROM, a fixed hard drive, and so on) as well as removable media (e.g., Flash memory, a removable hard drive, an optical disc, and so forth). The computer-readable media 506 may be configured in a variety of other ways as further described below.

Input/output interface(s) 508 are representative of functionality to allow a user to enter commands and information to computing device 502, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 502 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” “logic,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on and/or transmitted across some form of computer-readable media. The computer-readable media may include a variety of media that may be accessed by the computing device 502. By way of example, and not limitation, computer-readable media may include “computer-readable storage media” and “computer-readable transmission media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and nonvolatile, removable and non-removable media and/or storage devices implemented in a method or technology suitable for storage of information such as computer-readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable transmission media” may refer to a medium that is configured to transmit instructions to the hardware of the computing device 502, such as via a network. Computer-readable transmission media typically may transmit computer-readable instructions, data structures, program modules, or other data in a modulated data signal, such as carrier waves, data signals, or other transport mechanism. Computer-readable transmission media also include any information delivery media. The term “modulated data signal” means a signal that has one or more of its characteristics set or changed in such a manner as to encode information in the signal. By way of example, and not limitation, computer-readable transmission media include wired media such as a wired network or direct-wired connection, and wireless media such as acoustic, radio frequency (RF), infrared, and other wireless media.

As previously described, hardware elements 510 and computer-readable media 506 are representative of modules, programmable device logic and/or device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 510. The computing device 502 may be configured to implement particular instructions and/or functions corresponding to the software and/or hardware modules. Accordingly, implementation of a module that is executable by the computing device 502 as software may be achieved at least partially in hardware, e.g., through use of computer-readable storage media and/or hardware elements 510 of the processing system 504. The instructions and/or functions may be executable/operable by one or more articles of manufacture (for example, one or more computing devices 502 and/or processing systems 504) to implement techniques, modules, and examples described herein.

The techniques described herein may be supported by various configurations of the computing device 502 and are not limited to the specific examples of the techniques described herein. This functionality may also be implemented all or in part through use of a distributed system, such as over a “cloud” 514 via a platform 516 as described below.

The cloud 514 includes and/or is representative of a platform 516 for resources 518. The platform 516 abstracts underlying functionality of hardware (e.g., servers) and software resources of the cloud 514. The resources 518 may include applications and/or data that can be utilized while computer processing is executed on servers that are remote from the computing device 502. Resources 518 can also include services provided over the Internet and/or through a subscriber network, such as a cellular or Wi-Fi network.

The platform 516 may abstract resources and functions to connect the computing device 502 with other computing devices. The platform 516 may also be scalable to provide a corresponding level of scale to encountered demand for the resources 518 that are implemented via the platform 516. Accordingly, in an interconnected device embodiment, implementation of functionality described herein may be distributed throughout multiple devices of the system 500. For example, the functionality may be implemented in part on the computing device 502 as well as via the platform 516 which may represent a cloud computing environment 514.

The example systems and methods of the present disclosure overcome various deficiencies of known prior art devices. Other embodiments of the present disclosure will be apparent to those skilled in the art from consideration of the specification and practice of the disclosure contained herein. It is intended that the specification and examples be considered as example only, with a true scope and spirit of the present disclosure being indicated by the following claims.

What is claimed is:

1. A system, comprising:
a patient activity sensor;
one or more processors communicatively coupled to the patient activity sensor; and
one or more computer-readable media storing instructions that, when executed by the one or more processors, cause the one or more processors to perform operations comprising:
receiving, from the patient activity sensor, patient activity data indicative of activities of a patient;
determining a raw score for the patient based at least in part on the patient activity data, wherein determining the raw score comprises:
identifying a first component score from the patient activity data, the first component score associated with a first activity of the activities of the patient,
determining a transformation of the first component score associated with the first activity,
identifying a second component score from the activity data, the second component score associated with a second activity of the activities of the patient, and
combining the transformation of the first component score with the second component score associated with the second activity to generate the raw score;
determining that the raw score is greater than a score threshold;
generating an activity score based at least in part on determining that the raw score is greater than the score threshold; and
outputting the activity score to an electronic device separate from the one or more processors.

2. The system of claim 1, wherein the patient activity sensor comprises one or more of:
a hospital bed sensor;
an accelerometer of a device worn by the patient;
an overhead lift sensor;
a pressure-sensitive mat;
a camera;
a thermal sensor; and
an ultrasonic sensor.

3. The system of claim 1, wherein the activities of the patient comprise one or more of:
extremity movements;
assisted steps;
unassisted steps;
assisted turns in a hospital bed;
unassisted turns in the hospital bed;
assisted exits from the hospital bed;
unassisted exits from the hospital bed;
assisted ambulating;
unassisted ambulating;
sitting out of the hospital bed; and
sitting in the hospital bed.

4. The system of claim 1, the operations further comprising:
receiving, from a location sensor, an accelerometer, or a camera, an indication that a caregiver is within a threshold distance of the patient; and
determining, based at least in part on the indication, whether the activities of the patient are assisted or unassisted,
wherein at least one of the first component score or the second component score are based on whether the first activity or the second activity are assisted or unassisted.

5. The system of claim 1, wherein determining the raw score for the patient further comprises weighting one or more of the first component score or the second component score based at least in part on a first intensity of the first activity or a second intensity of the second activity.

6. The system of claim 5, wherein the operations further comprise receiving, from the electronic device, weight values to use in weighting the one or more of the first component score or the second component score.

7. The system of claim 5, wherein the operations further comprise receiving, from a machine-learned model, weight values to use in weighting the one or more of the first component score or the second component score.

8. The system of claim 1, the operations further comprising:
receiving a mobility protocol for the patient, the mobility protocol including mobility tasks associated with at least one of the first activity or the second activity;
determining, based at least in part on the patient activity data, that at least one task of the mobility tasks has been completed by the patient; and
outputting, to the electronic device, an indication that the at least one task of the mobility tasks has been completed by the patient.

9. The system of claim 1, the operations further comprising:
identifying, based at least in part on the patient activity data, at least one of the first activity or the second activity as being performed by the patient;
outputting, to the computing device, a notification of the at least one of the first activity or the second activity being performed by the patient; and
receiving, from the electronic device, a verification that the patient has performed the at least one of the first activity or the second activity,
wherein determining the raw score for the patient is based at least in part on receiving the verification.

10. The system of claim 1, the operations further comprising:
collecting the patient data over a first time period; and
determining, based at least in part on a user input or a detected activity associated with the patient, to pause collection of the patient data for a second time period,
wherein determining the raw score for the patient is exclusive of the second time period.

11. The system of claim 1, wherein combining the transformation of the first component score with the second component score comprises adding the transformation of the first component score to the second component score.

12. A method comprising:
receiving patient activity data indicative of activities of a patient;
determining a raw score for the patient based at least in part on the patient activity data, wherein determining the raw score comprises:
identifying a first component score from the activity data, the first component score associated with a first activity of the activities of the patient,
determining a transformation of a first component score associated with the first activity,
identifying a second component score from the activity data, the second component score associated with a second activity of the activities of the patient, and
combining the transformation of the first component score with a second component score associated with the second activity to generate the raw score;
determining that the raw score is greater than a score threshold;
generating an activity score based at least in part on determining that the raw score is greater than the score threshold; and
outputting the activity score to an electronic device.

13. The method of claim 12, further comprising:
identifying, based at least in part on the patient activity data, at least one of the first activity or the second activity as being performed by the patient;
outputting, to the computing device, a notification of the at least one of the first activity or the second activity being performed by the patient; and
receiving, from the electronic device, a verification that the patient has performed the at least one of the first activity or the second activity,
wherein determining the raw score for the patient is based at least in part on receiving the verification.

14. The method of claim 12, further comprising:
collecting the patient data over a first time period; and
determining, based at least in part on a user input or a detected activity associated with the patient, to pause collection of the patient data for a second time period,
wherein determining the raw score for the patient is exclusive of the second time period.

15. The method of claim 12, wherein combining the transformation of the first component score with the second component score comprises adding the transformation of the first component score to the second component score.

16. One or more computer-readable media storing instructions that, when executed by one or more processors, cause the one or more processors to perform operations comprising:
receiving patient activity data indicative of activities of a patient;
determining a raw score for the patient based at least in part on the patient activity data, wherein determining the raw score comprises:
identifying a first component score from the activity data, the first component score associated with a first activity of the activities of the patient,
determining a transformation of a first component score associated with the first activity,
identifying a second component score from the activity data, the second component score associated with a second activity of the activities of the patient, and
combining the transformation of the first component score with a second component score associated with the second activity to generate the raw score;
determining that the raw score is greater than a score threshold;
generating an activity score based at least in part on determining that the raw score is greater than the score threshold; and
outputting the activity score to an electronic device.

17. The one or more computer-readable media of claim 16, wherein determining the raw score for the patient further comprises weighting one or more of the first component score or the second component score based at least in part on a first intensity of the first activity or a second intensity of the second activity.

18. The one or more computer-readable media of claim 17, wherein the operations further comprise receiving, from the electronic device, weight values to use in weighting the one or more of the first component score or the second component score.

19. The one or more computer-readable media of claim 17, wherein the operations further comprise receiving, from a machine-learned model, weight values to use in weighting the one or more of the first component score or the second component score.

20. The one or more computer-readable media of claim 16, wherein at least a portion of the activity data is received from a hospital bed, the at least the portion of the activity data indicating a position of the patient, wherein the position comprises:
a side position;
a back position;
a prone position; or
a seated position.
